(19) United States
(12) Patent Application Publication
(10) Pub. No.: US 2017/0258040 A1
(43) Pub. Date: Sep. 14, 2017

(54) CLOTHING FOR TREATING FRACTURE OF PET DOG'S LEG

(71) Applicant: So Yoon BANG, Gangwon-do (KR)

(72) Inventor: So Yoon BANG, Gangwon-do (KR)

(21) Appl. No.: 15/310,177

(22) PCT Filed: Oct. 23, 2015
(86) PCT No.: PCT/KR2015/011256

§ 371 Date: Nov. 10, 2016

(30) Foreign Application Priority Data
Mar. 16, 2015 (KR) 10-2015-0035916

(51) Int. Cl. A01K 13/00; A61D 3/00; A61F 13/04; A61H 1/02; A61F 5/058

(52) U.S. Cl. CPC A01K 13/006; A61H 1/0218; A61F 5/0585; A61F 13/04; A61D 3/00; A61D 2003/006

(57) ABSTRACT

The present invention provides clothing for treating the fracture of a pet dog's leg, comprising: an outer cover surrounding the body of the pet dog; a traction belt which can extend along the circumference of the outer cover; and two leg insertion parts which are fixed to both ends of the traction belt, respectively, and into which forelegs or hind legs of the pet dog can be inserted, at least one of the two leg insertion parts being made of a plaster cast.

### TECHNICAL FIELD

The present invention relates to clothing for treating the fracture of a pet dog's leg, and more particularly, to clothing for treating the fracture of a pet dog' leg by applying appropriate restriction to the pet dog's leg.

### BACKGROUND ART

A plaster bandage is widely used to treat an injury such as a fracture. Since the plaster bandage hardens after it has been made wet in contact with a part to be treated, it can protect the corresponding part. For example, when a human arm or leg fractures, the plaster bandage is wound around the corresponding part and is made wet. After time elapses, the plaster bandage hardens while surrounding the arm or leg and thereby forms a cast. The plaster bandage which hardens as a cast puts restriction on the movement of the fractured part, and simultaneously, protects the fractured part from an external shock.

However, in the case of pet dogs, since they have low cognitive abilities, there is a problem that the plaster bandage is separated from a part to which it has been attached, unlike in the case of treating the fracture of people. In general, pet dogs are smaller than normal dogs and thus most of them are kept at home. However, if a pet dog has an accident when its owner goes out with it and thus fractures its leg, the pet dog is less likely to recognize pain in its leg even when a plaster bandage is wound, and there is a problem that the plaster bandage is easily released or separated from the dog's leg due to a lot of movement. Therefore, there is a problem that it is difficult to apply a plaster bandage and form a cast in a desired form. In addition, even simple treatment requires skills and thus there are problems that treatment costs increase and experts are required.

### DETAILED DESCRIPTION OF THE PRESENT DISCLOSURE

### Technical Objects

The present invention has been developed in order to solve the above-stated problems, and an object of the present invention is to provide clothing for treating a fracture, which can treat the fracture of a pet dog easily and cheaply, and can be prevented from being separated from a part to be treated and can be easily attached and detached, such that the clothing can be easily managed, and by which the pet dog can receive treatment without resisting strongly

### Technical Solving Means

According to the present invention, there is provided clothing for treating the fracture of a pet dog's leg including: an outer cover surrounding the body of the pet dog; a traction belt which extends along the circumference of the outer cover 11; and two leg insertion parts which are fixed to both ends of the traction belt, respectively, and into which forelegs or hind legs of the pet dog are inserted, at least one of the two leg insertion parts being made of a plaster bandage.

According to an aspect of the present invention, the outer cover may have a connection part which extends from a first side through which a pet dog's head is exposed to a second side through which a pet dog's tail is exposed, and the outer cover may have a hole formed thereon to allow the pet dog's leg to be inserted therethrough.

According to another aspect of the present invention, the leg insertion parts may be fixed and maintained at one end of the traction belt, whereas the leg insertion parts 12 and 13 may be maintained to be removable at the other end of the traction belt 15.

According to another aspect of the present invention, the traction belt may be formed of a metal flat plate, such that the traction belt maintains its bent state to fit on the circumference of the body of the pet dog, and the traction belt may be inserted through a belt passing part provided on the outer cover.

According to another aspect of the present invention, an inner edge of each of the leg insertion parts may be connected to an outer edge of the hole of the outer cover.

### Advantageous Effect

The clothing for treating the fracture of the pet dog's leg according to the present invention can appropriately restrict movement of the pet dog, and accordingly, can protect the fractured leg of the pet dog and can be prevented from being released from the pet dog and can be fixed, such that the fractured part can be rapidly recovered. In addition, the clothing for treating the fracture of the pet dog's leg according to the present invention can be used by a normal user in addition to a vet, and also, there is an advantage that the pet dog can be covered with the clothing for treating the fracture of the pet dog's leg without resisting. In addition, since the clothing for treating the fracture of the pet dog's leg according to the present invention can be cheaply configured, many social problems caused by abandoned dogs in streets or parks because dog owners cannot bear the expensive costs of treatments of injured dogs can be solved.

### BRIEF DESCRIPTION OF THE DRAWINGS

FIG. 1 is a schematic view to illustrate clothing for treating the fracture of a pet dog's leg according to the present invention.

### BEST MODE FOR EMBODYING THE INVENTION

Hereinafter, the present invention will be described in detail with reference to an embodiment illustrated in the accompanying drawing.

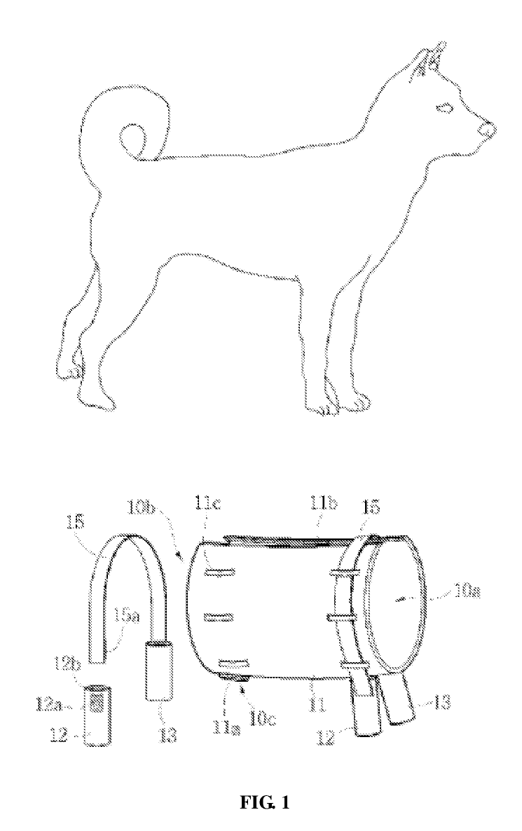

FIG. 1 is an exploded perspective view showing a part of clothing for a pet dog, which is provided with a plaster bandage according to an exemplary embodiment of the present invention.

Referring to the drawing, the clothing for the pet dog according to the present invention includes an outer cover 11 surrounding the body of the pet dog, a traction belt 15 which can extend along the circumference of the outer cover 11, and two leg insertion parts 12 and 13 which are fixed to both ends of the traction belt 15, at least one of the leg insertion parts 12 and 13 being made of a plaster bandage.

The outer cover 11 is formed to surround the body of the pet dog, and the pet dog's neck and head may be exposed to the outside through a first side 10a of the outer cover 11, and the pet dog's tail may be exposed to the outside through a second side 10b of the outer cover 11. Four holes 10c are formed on the bottom of the outer cover 11 to allow the pet dog's feet to be exposed to the outside of the outer cover 11.

The outer cover 11 is provided with a connection part 11b extending from the first side 10a of the outer cover 11 to the second side 10b in the longitudinal direction of the outer cover 11 and formed to be separated. The connection part 11b is formed of a zipper or a Velcro tape, such that a user can open and close the outer cover 11 through the connection part 11b. To cover the pet dog with the outer cover 11, the user completely opens the connection part 11b and inserts the pet dog's legs into the holes 10c, and exposes the head and the tail through the first side 10a and the second side 10b, respectively. Thereafter, the user connects the connection part 11b using the zipper or the Velcro tape constituting the connection part of the outer cover 11, such that the outer cover 11 surrounds the body of the pet dog.

A belt passing part 11c is formed on the surface of the outer cover 11 to allow the traction belt 15 to be inserted thereinto and to pass therethrough. Preferably, a plurality of belt passing parts 11c may be formed such that the traction belt 15 can be stably maintained on the surface of the outer cover 11. The belt passing part 11c is disposed at a location corresponding to the two forelegs or hind legs of the pet dog along the circumference of the outer cover 11. The traction belt 15 may be inserted into the belt passing part 11c or may be pulled through the belt passing part 11c. For example, the belt passing part 11c may be formed by attaching an annular member to the surface of the outer cover 11 or by forming an insertion passing hole by cutting a part of the outer cover 11.

In another example (not shown), instead of providing the belt passing part 11c, the traction belt 15 and the outer cover 11 may be attached to and detached from each other through a Velcro tape. That is, a Velcro tape is bonded to an entirety or a part of the surface of the outer cover 11, and a Velcro tape is bonded to a corresponding surface of the traction belt 15, such that the traction belt 15 is fixed to a certain surface of the outer cover 11.

The traction belt 15 has the leg insertion parts 12 and 13 which are formed at both ends thereof and into which the pet dog's legs are inserted. In the example illustrated in the drawing, the leg insertion parts for the forelegs are attached to the outer cover 11, and the leg insertion parts 12 and 13 for the hind legs are separated from the outer cover 11. In practice, it is not necessary to surround all of the pet dog's legs with the leg insertion parts, and for example, only the two forelegs or only the two hind legs may be surrounded by the leg insertion parts. Alternatively, only one of the four legs may be surrounded by the leg insertion part.

The traction belt 15 may be formed of various materials. For example, the traction belt 15 may be formed of a fabric or metal or flexible synthetic resin. In the preferred embodiment illustrated in the drawing, the traction belt 15 may be formed of a bendable metal flat plate. The metal traction belt 15 may be inserted into the belt passing part 11c of the outer cover 11, and may be deployed and bent on the circumference of the body of the pet dog, and maintains its bent state after it has been bent. Accordingly, the traction belt 15 can keep pulling the leg insertion parts 12 and 13 attached to both ends thereof without applying excessive force to the body of the pet dog.

In the example illustrated in the drawing, the traction belt 15 is provided with the removable leg insertion part 12 formed at one side thereof, and the fixed leg insertion part 13 formed at the other side thereof. For example, the removable leg insertion part 12 may be attached to or detached from the traction belt 15 using a Velcro tape.

To maintain the leg insertion part 12 to be removable from the traction belt 15, a first Velcro tape member 15a is attached to one side of the traction belt 15 and a second Velcro tape member 12a is attached to a corresponding side of the leg insertion part 12. Accordingly, the first Velcro tape member 15a and the second Velcro tape member 12a may be detached from or attached to each other, and accordingly, the leg insertion part 12 may be detached from or attached to the traction belt 15. The other leg insertion part 13 fixed to the other end of the traction belt 15 may maintain its fixing state to the traction belt 15 by means of an adhesive or the like, for example. In another example (not shown), it will be understood that both of the two leg insertion parts 12 and 13 are configured to be removable from the traction belt 15.

The traction belt 15 may be pulled by changing the mutual connection location of the first Velcro tape member 15a and the second Velcro tape member 12a. For example, in a state in which one of the two hind legs of the pet dog is inserted into the fixed leg insertion part 13 and the other leg is inserted into the removable leg insertion part 12, the traction belt 15 is made to pass through the belt passing part 11c of the outer cover 11. At the same time, when the first Velcro tape member 15a and the second Velcro tape member 12a are attached to each other while pulling the traction belt 15, the tension state of the traction belt 15 can be maintained.

Preferably, the entirety of the leg insertion parts 12 and 13 may be formed in a cylindrical shape so as to allow the pet dog's legs to be inserted thereinto. One of the two leg insertion parts 12 and 13 may be made of a plaster bandage or both the two leg insertion parts 12 and 13 may be made of a plaster bandage. As well known, the plaster bandage hardens after it has been made wet, and accordingly, a fractured part wound by the plaster bandage can be stably protected and maintained.

Preferably, an inner Velcro tape member 12b may be provided on the inner edge of the upper portion of each of the leg insertion parts 12 and 13, and an outer Velcro tape member 11a may be provided on the outer edge of the hole 10c of the outer cover 11 corresponding to the inner edge of the upper portion of each of the leg insertion parts 12 and 13. Accordingly, after the leg insertion parts 12 and 13 fit on both legs of the pet dog, the mutual connection state between the outer cover 11 and the leg insertion parts 12 and 13 can be maintained through the inner and outer Velcro tape members 12b and 11a.

Hereinafter, a method of using the clothing for the pet dog according to the present invention will be explained briefly.

Before the clothing for the pet dog is used, the outer cover 11, the traction belt 15, and the leg insertion parts 12 are separated. If there is a need to restrict some movement of the pet dog because of the fracture of the pet dog's leg or the like, the pet dog is covered with the outer cover 11 first. The outer cover 11 surrounds the body of the pet dog by opening the connection part 11b, inserting the pet dog's legs into the holes 10c, and then closing the connection part 11b. In this case, the pet dog's neck and head are exposed through the first side 10a and the pet dog's tail is exposed through the second side 10b.

Thereafter, the pet dog's legs are inserted into the leg insertion parts 12 and 13. In this case, the pet dog's fractured leg is inserted into the leg insertion part made of the plaster bandage. In the example illustrated in the drawing, when the leg insertion part 12 is made of the plaster bandage, the user inserts the pet dog's leg which does not fracture into the leg insertion part 13, and inserts the opposite leg which fractures into the leg insertion part 12. Next, the inner Velcro tape member 12b provided on each of the leg insertion parts 12 and 13 is attached to the outer Velcro tape member 11a provided on the hole 10c of the outer cover 11.

In this case, the traction belt 15 is inserted through the belt passing part 11c and pulled, and the first Velcro tape member 15a provided at one end of the belt passing part 11c is attached to the corresponding second Velcro tape member 12a provided on the leg insertion part 12. The traction state of the traction belt 15 can be controlled by changing the relative location between the first Velcro tape member 15a and the second Velcro tape member 12a.

Thereafter, a part or entirety of the leg insertion part 12 may be made to harden by making a part of the leg insertion part 12 made of the plaster bandage wet. While the plaster bandage hardens, the user may apply appropriate press to the plaster bandage to suit to the leg shape of the pet dog, such that the leg insertion part 12 can appropriately restrict the movement of the pet dog's leg when the plaster bandage hardens.

As described above, only one of the leg insertion parts 12,13 may be made of the plaster bandage or both of them may be made of the plaster bandage. In addition, only the forelegs of the pet dog may be inserted into the leg insertion parts 12, 13 or only the hind legs of the pet dog may be inserted into the leg insertion parts. Alternatively, it should be understood that all of the four legs may be inserted into the leg insertion parts.

What is claimed is:

1. Clothing for treating the fracture of a pet dog's leg, comprising:
an outer cover 11 surrounding the body of the pet dog;
a traction belt 15 which extends along the circumference of the outer cover 11; and
two leg insertion parts 12 and 13 which are fixed to both ends of the traction belt 15, respectively, and into which forelegs or hind legs of the pet dog are inserted, at least one of the two leg insertion parts 12 and 13 being made of a plaster bandage.

2. The clothing of claim 1, wherein the outer cover 11 has a connection part 11b which extends from a first side 10a through which a pet dog's head is exposed to a second side 10b through which a pet dog's tail is exposed, and the outer cover 11 has a hole 10c formed thereon to allow the pet dog's leg to be inserted therethrough.

3. The clothing of claim 1, wherein the leg insertion parts 12 and 13 are fixed and maintained at one end of the traction belt 15, whereas the leg insertion parts 12 and 13 are maintained to be removable at the other end of the traction belt 15.

4. The clothing of claim 1, wherein the traction belt 15 is formed of a metal flat plate, such that the traction belt 15 maintains its bent state to fit on the circumference of the body of the pet dog, and
wherein the traction belt 15 is inserted through a belt passing part 11c provided on the outer cover 11 or is maintained by Velcro tapes bonded to the traction belt 15 and the outer cover 11.

5. The clothing of claim 2, wherein an inner edge of each of the leg insertion parts 12 and 13 is connected to an outer edge of the hole 10c of the outer cover 11.
